(19) United States
(12) Patent Application Publication
(10) Pub. No.: US 2015/0031019 A1
(43) Pub. Date: Jan. 29, 2015

(54) Computer Assisted Sperm Profile Analysis and Recognition

(71) Applicant: CELLCURA AS, Skien (NO)

(72) Inventors: Bent Nordbo, Oslo (NO); Jayant Mehta, Morden (GB); Lesley Hutchins, Madison, WI (US); Gerd Staume, Oslo (NO)

(73) Assignee: CELLCURA AS, Skien (NO)

(21) Appl. No.: 14/211,171

(22) Filed: Mar. 14, 2014

Related U.S. Application Data
(60) Provisional application No. 61/784,758, filed on Mar. 14, 2013.

(51) Int. Cl. G01N 33/50; G06F 19/22

(52) U.S. Cl. CPC G01N 33/5005; G06F 19/22
USPC 435/6.1; 702/19

(57) ABSTRACT

The invention described herein relates to a device that will discriminate between normal sperm, and sperm that can look morphologically normal, but with >5% DNA fragmentation using an expert system that will allow the embryologist to select morphologically and genetically normal sperm.

### CROSS-REFERENCE TO RELATED APPLICATIONS

This application claims the benefit of U.S. Provisional Application No: 61/784,758 filed Mar. 14, 2013, the disclosure of which is expressly incorporated by reference in its entirety.

### BACKGROUND OF THE INVENTION

1. Field of the Invention

The present invention relates generally to the fields of cell biology, embryology, fertility, sperm morphology, and particularly to selecting morphologically and genetically normal sperm from a semen or other biological sample.

2. Description of Related Art

The fertility rates in all Organization for Economic Co-Operation and Development (OECD) countries have declined to record low levels over the past decades. While the average fertility rate was 2.7 children per woman in childbearing age in 1970, the fertility rate had declined to 1.6 in 2002. This is well below the replacement fertility rate of 2.1 children per woman that would secure a stable population. The increasing use of assisted reproductive techniques also indicates that infertility is a matter of growing concern. Infertility affects 15% of all couples. While it is well-known that women postponing motherhood have reduced fertility rates, men's reproductive health on a couple's fertility has often been overlooked. Abnormal sperm behavior is an important indicator for male infertility and there is currently a growing interest for understanding the kinetics and dynamics of swimming spermatozoa and to search for better methods to select the best suited spermatozoon for assisted reproductive techniques.

To substantiate that DNA aberrations can be recognized and classified by observing the physical and kinetic attributes of a single spermatozoon, extensive studies of the behavior of genetically normal and abnormal cells is required. Assisted reproductive techniques (ART) utilize both the female and male gametes to achieve a clinical pregnancy. In a traditional in vitro fertilization (IVF) procedure, considerable attention is given to pick the right egg for insemination and optionally for freezing and/or donation. However, when it comes to sperm selection, especially in case of intra-cytoplasmic sperm injection (ICSI), the sperm with a higher percentage of DNA fragmentation or damage are not separated from those that have intact DNA; in fact, spermatozoa with normal morphological appearance but with fragmented DNA could be selected for actual fertilization of oocytes. Therefore, it is probable that the sperm injected based on its morphology, cannot be genetically normal.

Computer Assisted Sperm Analysis (CASA) systems are often used in sperm selection procedures. This process takes 10-15 minutes in total, and the end result delivers a population profile of sperm that swim in different ways, with the percentage of progressive motile population being the most important selection criterion for potential successful fertilization. To obtain more detailed information, sperm can be fixed and stained histologically to reveal specific morphological attributes and in particular specific abnormalities that will impair the ability of the sperm to fertilize the oocyte. Like CASA, sperm morphology gives a profile of the different populations in the semen sample and ultimately the percentage of statistically morphologically normal sperm in the semen sample. Whether there is any correlation between the percentage DNA fragmentation of sperm and percentage of morphologically abnormal sperm remains to be qualified.

Research has shown that between 20% and 66% of normal-looking sperm from infertile men contains DNA damage, indicating that spermatozoa with normal morphological appearance can have damaged or fragmented DNA that makes the sperm infertile. In many European countries at least 20% of young men exhibit semen quality parameters below the World Health Organization (WHO) reference values and this will most likely affect their fertility. In addition, the percentage of sperm with damaged DNA, due to environmental stress, Reactive Oxygen Species (ROS) etc., influence the fertility. DNA fragmentation therefore, also contributes to infertility and should be assessed as a part of the basic semen analysis.

Software applications that provide accurate and consistent morphological analysis of oocytes and embryos designed for research and routine scoring procedures are currently available. In addition a system that measures sperm concentration in sperm samples and performs an objective and detailed analysis of sperm motility according to the WHO standard is also currently available. However, research on correlation between sperm attributes and DNA integrity is required by the industry and can undoubtedly increase the competence levels in the involved companies and bring about new skills and tools that will strengthen its position in the assisted reproductive techniques (ART) marketplace.

The objective of currently available different methods for sperm selection is to assist the embryologist to select the spermatozoon with the highest probability of successful fertilization and clinical pregnancy. The major disadvantage of these methods is that the analyzed sperm samples cannot be used for fertilization. Consequently, the embryologist cannot be certain of the quality of the spermatozoon finally selected for actual treatment. As such, to mitigate this uncertainty, the inventors have developed Computer Aided Sperm Analysis and Recognition system (CASPAR) to non-invasively characterize, in real time, the true relationship between motility, individual morphology (cell topography and internal structure) and percentage of DNA damage/fragmentation of an individual's live spermatozoon.

### SUMMARY OF THE INVENTION

It is against the above background that the present invention provides certain advantages and advancements over the prior art.

To mitigate uncertainty in the prior art concerning the quality of spermatozoa selected for, inter alia, IVF, set forth herein is a Computer Aided Sperm Analysis and Recognition system (CASPAR) that non-invasively characterizes, in real time, the true relationship between motility, individual morphology (cell topography and internal structure) and percentage of DNA damage/fragmentation of an individual's live spermatozoon.

Although this invention disclosed herein is not limited to specific advantages or functionality, the invention provides a pattern recognition system that can enable an embryologist to, non-invasively and in real time, identify and select a single spermatozoon with high motility, normal morphology and high DNA integrity.

The methods and devices of the invention can provide a pattern recognition system that can enable an embryologist to instantly and non-invasively identify and select a single spermatozoon with high motility, normal morphology and high DNA integrity for use in the fertilization process, and thus significantly increasing the probability of a viable clinical pregnancy. The instant system can provide: 1) an affordable, non-invasive technique that is both simple and quick such that the spermatozoon under analysis can be used for actual treatment; 2) real-time inquiry against a reference database for recognition of sperm with motility and morphology patterns that matches profiles with high DNA integration; 3) reduce the probability of DNA fragmentation in the selected sperm and increases the success rates for a clinical pregnancy; 4) reduce psychological strain on the infertile couples; and 5) reduce costs for the society where treatment of infertility.

Although this invention disclosed herein is not limited to specific advantages or functionality, the invention provides a method for identifying and selecting high motility, normal morphology and high DNA integrity spermatozoa comprising: (a) receiving a sperm sample; (b) determining one or more characteristics of each spermatozoon in the sperm sample; and (c) determining a sperm kinetic profile of the spermatozoon in the sperm sample; (d) and associating the sperm kinetic profile with an index set, wherein the index includes information indicative of an identity of the sperm kinetic profile.

In some aspects, determining the sperm kinetic profile comprises determining a fingerprint value at one or more landmarks of the spermatozoon, wherein each landmark represents a distinctive and reproducible component of the spermatozoon, and wherein the fingerprint value represents a number of features of the spermatozoon at the distinctive and reproducible components of a given spermatozoon.

In some aspects, the spermatozoon has 5% or less DNA fragmentation.

In some aspects, the spermatozoon is from Homo sapiens.

In some aspects, the spermatozoon identified to have a preferred kinetic sperm profile is selected for use in a fertilization process.

The invention further provides a hardware platform device with an integrated tracking and machine vision system and a pattern matching algorithm that matches the input from the machine vision system with the pattern matching algorithm based on a set of pre-defined patterns in a reference database.

In some aspects, the set of pre-defined patterns recognize sperm with motility and morphology patterns that match profiles with high DNA integrity for use in the fertilization process, and thus significantly increasing the probability of a viable clinical pregnancy.

In some aspects, the hardware platform device set forth herein comprises a buffer configured to receive a sperm sample; a processor; and instructions stored in memory and executable by the processor to cause the processor to perform functions comprising: receiving the sperm sample into the buffer; storing the received sperm sample; and performing a content identification of each spermatozoon in the buffer to determine a kinetic sperm profile of the received sperm sample.

The invention further provides a hardware platform device comprising a handheld system, a microfluidic chip and software and pattern matching algorithms.

In some aspects, the microfluidic chip and software can be used for semen analysis.

In some aspects, the microfluidic chip and software can be used for oocyte analysis.

In some aspects, the microfluidic chip and software can be used for embryo scoring.

In some aspects, the hardware platform device set forth herein comprises a communication device for transfer of data to cloud applications.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings.

FIG. 1 shows a test design for phase 1 where the reference database is created.

FIG. 2 shows a test design for the multicenter prospective randomized study.

FIG. 3 shows a flow diagram of an example method of the invention for recognizing a spermatozoon in a sperm sample.

### DETAILED DESCRIPTION OF THE INVENTION

All publications, patents and patent applications cited herein are hereby expressly incorporated by reference for all purposes.

Unless otherwise defined, all technical and scientific terms used herein have the same meaning as commonly understood by one of ordinary skill in the art to which the invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the invention will be apparent from the following detailed description. Applicants reserve the right to alternatively claim any disclosed invention using the transitional phrase “comprising,” “consisting essentially of,” or “consisting of,” according to standard practice in patent law.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

In one aspect, the invention provides a pattern recognition system that can enable an embryologist to identify and select a single spermatozoon with high motility, normal morphology and high DNA integrity. In some embodiments, the method for identification and selection of high motility, normal morphology and high DNA integrity spermatozoa comprises: (a) receiving a sperm sample; (b) determining one or more characteristics of each spermatozoon in the sperm sample; and (c) determining a sperm kinetic profile of the spermatozoon in the sperm sample; (d) and associating the sperm kinetic profile with an index set, wherein the index includes information indicative of an identity of the sperm kinetic profile. Furthermore, determining the sperm kinetic profile comprises determining a fingerprint value at one or more landmarks of the spermatozoon, wherein each landmark represents a distinctive and reproducible component of the spermatozoon, and wherein the fingerprint value represents a number of features of the spermatozoon at the distinctive and reproducible components of a given spermatozoon. In such a method, the spermatozoa identified to have a preferred kinetic sperm profile are selected for use in a fertilization process.

DNA integrity can be measured by any number of ways as recognized by one skilled in the art. Non-limiting examples of assays for the detection of DNA damage or DNA fragmentation in eukaryotic cells include: single cell gel electrophoresis assay (COMET assay); terminal deoxynucleotidyl transferase dUTP nick end labeling assay (TUNEL assay); Sperm Chromatin Structure Assay (SCSA) and the HALOSPERM® test.

In a further aspect, the invention provides instruments that can discriminate between normal sperm (i.e., sperm with no or <5% DNA fragmentation), and sperm that can look morphologically normal, but with >5% DNA fragmentation. Such an expert instrument system allows an embryologist to classify and characterize quantitatively different vacuoles observed in the head of the sperm with the percentage of DNA fragmentation, thus, selecting morphologically and genetically normal sperm that demonstrate a higher rate of successful clinical pregnancies.

In a further aspect, the invention provides hardware platform devices with an integrated tracking and machine vision system and a pattern matching algorithm that matches the input from the machine vision system with the pattern matching algorithm based on a set of pre-defined patterns in a reference database.

In another aspect, the invention provides hardware platform devices, wherein the set of pre-defined patterns recognize sperm with motility and morphology patterns that match profiles with high DNA integrity for use in the fertilization process, and thus significantly increases the probability of a viable clinical pregnancy.

In yet another aspect, the invention provides methods for identifying and selecting high motility, normal morphology and high DNA integrity spermatozoon comprising: (a) receiving a sperm sample; (b) determining one or more characteristics of each spermatozoa in the sperm sample; and (c) determining a sperm kinetic profile of the spermatozoa in the sperm sample; (d) and associating the sperm kinetic profile with an index set, wherein the index includes information indicative of an identity of the sperm kinetic profile. In an embodiment, determining a sperm kinetic profile comprises determining a fingerprint value at one or more landmarks of the spermatozoa, wherein each landmark represents a distinctive and reproducible component of the spermatozoa, and wherein the fingerprint value represents a number of features of the spermatozoa at the distinctive and reproducible component of a given spermatozoa; providing the index set comprising the fingerprint value at the landmark. In an embodiment, a preferred sperm kinetic profile is one that correlates with increased probability of fertilization and successful pregnancy.

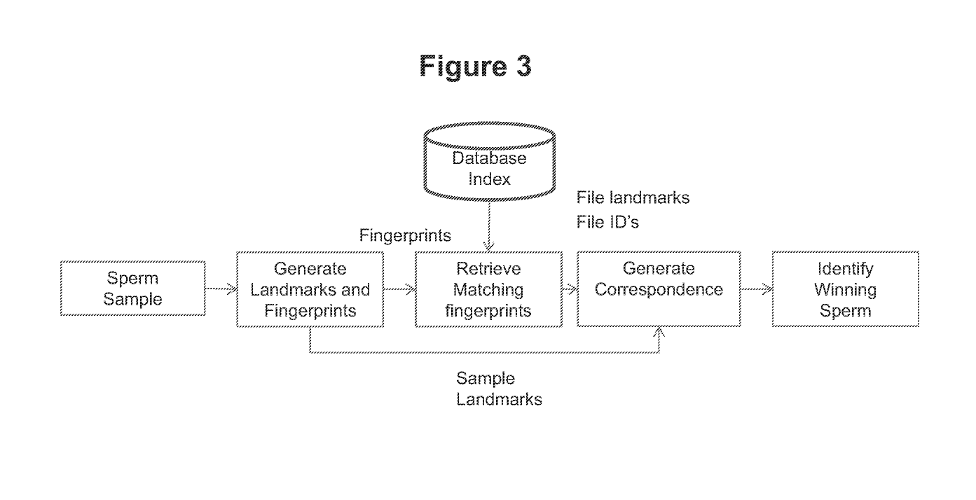

In an embodiment, the method identifies a winning spermatozoon; a spermatozoon whose relative locations of characteristic fingerprints most closely match the relative locations of the same fingerprints of the database index (see FIG. 3). After a sperm sample is captured, landmarks and fingerprints are computed. Landmarks occur at particular morphological components, e.g., head, nucleus, centriole, neck or tail, within the spermatozoon. The location within the spermatozoon of the landmarks is preferably determined by the spermatozoon itself, i.e., is dependent upon each spermatozoon, and is reproducible. That is, the same landmarks are computed for the same component each time the process is repeated. For each landmark, a fingerprint characterizing one or more features of the spermatozoon at or near the landmark is obtained. The nearness of a feature to a landmark is defined by the fingerprinting method used. In some cases, a feature is considered near a landmark if it clearly corresponds to the landmark and not to a previous or subsequent landmark. In other cases, features correspond to multiple adjacent landmarks. For example, fingerprints can be spermatozoon trajectory or velocity parameters, and image fingerprints can be pixel RGB values.

The sample fingerprints are used to retrieve sets of matching fingerprints stored in a database index, in which the matching fingerprints are associated with landmarks and identifiers of a set of spermatozoa with known kinetic and DNA fragmentation attributes (i.e., correlations between spermatozoon's physical behavior and morphology and successful fertilization and pregnancy rates and spermatozoa with high DNA integrity demonstrate a unique and recognizable linear progressive movement pattern, unique curvilinear velocity and lateral head displacement measurements). The set of retrieved file identifiers and landmark values are then used to generate correspondence pairs containing sample landmarks and retrieved file landmarks at which the same fingerprints were computed. The resulting correspondence pairs are then sorted by spermatozoon identifier, generating sets of correspondences between sample landmarks and database landmarks for each applicable spermatozoon. Each set is scanned for alignment between the database landmarks and sample landmarks. That is, linear correspondences in the pairs of landmarks are identified, and the set is scored according to the number of pairs that are linearly related. A linear correspondence occurs when a large number of corresponding sample locations and file locations can be described with substantially the same linear equation, within an allowed tolerance. For example, if the slopes of a number of equations describing a set of correspondence pairs vary by 5%, then the entire set of correspondences is considered to be linearly related. Of course, any suitable tolerance can be selected. The identifier of the set with the highest score, i.e., with the largest number of linearly related correspondences, is the winning spermatozoon, which is located and returned. Recognition can be performed in essentially real time, even with a very large database. That is, a sperm sample can be recognized as it is being processed, with a small time lag. The method can identify a spermatozoon based on 1 or 2 or 3 or more components. In a preferred embodiment, the landmarking and fingerprinting analysis, is carried out in real time as the sperm sample is being processed. Database queries are carried out as spermatozoon sample fingerprints become available, and the correspondence results are accumulated and periodically scanned for linear correspondences. Thus all of the method steps occur simultaneously and not in the sequential linear fashion suggested in FIG. 3. Note that the method is in part analogous to a text search engine: a user submits a query sperm sample, and a matching spermatozoon indexed in the spermatozoon database is returned.

In some embodiments, the inventive methods are typically implemented as software running on a computer system, with individual steps most efficiently implemented as independent software modules. Thus a system implementing the present invention can be considered to comprise a landmarking and fingerprinting object, an indexed database, and an analysis object for searching the database index, computing correspondences, and identifying the winning spermatozoon. In the case of sequential landmarking and fingerprinting, the landmarking and fingerprinting object can be considered to be distinct landmarking and fingerprinting objects. Computer instruction code for the different objects is stored in a memory of one or more computers and executed by one or more computer processors. In one embodiment, the code objects are clustered together in a single computer system, such as an Intel-based personal computer, workstation or other computing device. In another embodiment, methods are implemented by a networked cluster of central processing units (CPUs), in which different software objects are executed by different processors in order to distribute the computational load. Alternatively, each CPU can have a copy of all software objects, allowing for a homogeneous network of identically configured elements. In this latter configuration, each CPU has a subset of the database index and is responsible for searching its own subset of media files.

In an embodiment, a searchable spermatozoa database index can be constructed and implemented on a computer system or hardware platform devices set forth herein. As used herein, a database is any indexed collection of data, and is not limited to commercially available databases. In the database index, related elements of data are associated with one another, and individual elements can be used to retrieve associated data. The spermatozoa database index contains an index set for spermatozoa in the selected collection. Each set of spermatozoa also has a unique identifier, spermatozoon identifier. The spermatozoon database index can be very large, containing indices for millions or even billions of spermatozoa. In an embodiment, landmarks are first computed, and then fingerprints are computed at or near the landmarks. As will be apparent to one of average skill in the art, alternative methods can be devised for constructing the database index. In particular, many of the steps are optional, but serve to generate a database index that is more efficiently searched. While searching efficiency is important for real-time spermatozoon identification from large databases, small databases can be searched relatively quickly. To index the database, each spermatozoon identifier in the collection is subjected to a landmarking and fingerprinting analysis that generates an index set for each spermatozoon. Landmarks occur at specific components of the spermatozoon, while fingerprints characterize the spermatozoon at or near a particular landmark. Thus, in an embodiment, each landmark for a particular spermatozoon is unique, while the same fingerprint can occur numerous times within a single spermatozoon or multiple spermatozoa.

In yet another aspect, the invention relates to hardware platform devices comprising a handheld system, a microfluidic chip and software and pattern matching algorithms. In some embodiments, the microfluidic chip and software can be used for semen analysis, oocyte analysis or embryo scoring. In other embodiments, the device can integrate one or several laboratory functions on a single microfluidic chip of only millimeters to a few square centimeters in size. The device can handle small fluid volumes down to less than a milliliter, less than a microliter or less than a picoliter. A microfluidic chip can be a set of micro-channels etched or molded into a material (e.g., glass, silicon or polymer such as polydimethylsiloxane). The micro-channels forming the microfluidic chip can be connected together so as to achieve a desired function (e.g., mix, pump, redirect and/or allow chemical reactions in a cell of the chip). This network of micro-channels trapped in the microfluidic chip can be connected to the outside by inputs and outputs pierced through the chip, as an interface between the macro- and micro-world. It is through these holes that the liquids can be injected and removed from the microfluidic chip (through tubing, syringe adapters or even free holes in the chip). Typically fluids are moved, mixed, separated or otherwise processed. Numerous applications employ passive fluid control techniques like capillary forces. In some embodiments, external actuation means can be additionally used for a directed transport of the sample. In other embodiments, rotary drives can apply centrifugal forces for the fluid transport on the passive chips. Active microfluidics refers to the defined manipulation of the working fluid by active (micro) components as micro-pumps or micro-valves. Micro-pumps can supply fluids in a continuous manner or are used for dosing. Micro-valves can determine the flow direction or the mode of movement of pumped liquids. Often processes which are normally carried out in a lab are miniaturized on a single microfluidic chip in order to enhance efficiency and mobility as well as reduce sample and reagent volumes.

In other embodiments, the hardware platform devices can comprise communication devices for transfer of data to cloud applications. Computer networking or cloud computing can involve a large number of computers connected through a communication network such as the Internet, and can provide the ability to run a program or application on many connected computers at the same time. A cloud infrastructure can be operated for a single organization, whether managed internally or by a third-party and hosted internally or externally. A cloud application can be an application program that functions in the cloud, with some characteristics of a pure desktop application and some characteristics of a pure Web application. While a desktop application resides entirely on a single device at the user's location, a Web application is stored entirely on a remote server and can be delivered over the Internet through a browser interface. The user can cache data locally, enabling full offline mode when desired. A cloud application, unlike a Web application, can be used in situations where wireless devices are not allowed, because the application can function even when the Internet connection is disabled. In addition, cloud apps can provide some functionality even when no Internet connection is available for extended periods. Cloud applications can provide fast responsiveness and can work offline. Cloud applications need not permanently reside on the local device, but they can be easily updated online. Cloud applications are therefore under the user's constant control, yet they need not always consume storage space on the user's computer or communications device. A cloud application can offer all the interactivity of a desktop application along with the portability of a Web application.

### EXAMPLES

The Examples that follow are illustrative of specific embodiments of the invention, and various uses thereof. They are set forth for explanatory purposes only, and are not to be taken as limiting the invention.

### Example 1

### Computer Aided Sperm Analysis and Recognition System (CASPAR)

The system can non-invasively and in real time, discriminate between normal sperm (i.e., sperm with no or <5% DNA fragmentation), and sperm that can look morphologically normal, but with >5% DNA fragmentation. Such a system can allow an embryologist to classify and characterize quantitatively (i.e., generate a kinetic sperm profile) different vacuoles observed in the head of the sperm with the percentage of DNA fragmentation, thus, selecting morphologically and genetically normal sperm.

The system can further help to 1) assess and categorize details (according to WHO criteria) in a spermatozoon's movement pattern, morphological attributes and DNA integrity; 2) establish a database and algorithms, where correlation between sperm trajectory and velocity parameters are represented by a set of mathematical expressions (“fingerprints”), for pattern recognition (CASPAR); 3) assess details in an embryo's development between spermatozoa selected with the CASPAR method of the invention and spermatozoa selected with traditional CASA methods; 4) complete a clinical study to verify the rate of successful pregnancies; 5) compare behavior differences between fresh sperm and sperm that has been frozen and thawed; and 6) develop a scanning device for real-time analysis of semen samples.

The system can show that normal spermatozoa with high DNA integrity demonstrate a unique and recognizable linear progressive movement pattern, unique curvilinear velocity and lateral head displacement measurements, when compared with spermatozoa with >5% DNA fragmentation. The system can further demonstrate that spermatozoa selected (based on their kinetic sperm profile) by the methods of the invention (e.g., CASPAR) produce significantly higher rates of clinical pregnancies than spermatozoa chosen using traditional sperm selection techniques.

A central question to be answered is how DNA fragmentation or damage within the nucleus of individual spermatozoon affects its trajectory, curvilinear progression, lateral head displacement measurements, and fertilization capabilities. There is sufficient evidence that points to positive correlations between spermatozoa's physical behavior and morphology and pregnancy rates. Correspondingly, research points to a successful clinical pregnancy being achieved with “genetically normal” gametes. However, since there is no single method that can identify a “genetically normal” sperm, for use in ART treatments, especially for “Intra Cytoplasmic Sperm Injection” (ICSI), development of a method/product which aids the embryologist in selecting the “optimal sperm” will be helpful.

As noted above, it is also established that sperm DNA integrity is essential in fertilization and successful pregnancy. Routine semen analysis gives an approximate evaluation of the functional competence of spermatozoa, but does not always reflect the quality of sperm DNA. Therefore, the evaluation of sperm DNA integrity, in addition to routine sperm parameters like motility and morphology, can add further information on the quality of spermatozoa and reproductive potential of males. Consequently, by inspecting a live cell through a monitoring device, it can be possible to discriminate between normal sperm with no or <5% DNA fragmentation (“genetically normal”) and sperm that can look morphologically normal but with >5% DNA fragmentation.

The testing and evaluation of the system can be divided into 2 phases. In Phase 1, a reference database is created using semen samples from 100 patients attending a fertility clinic. Each patient has his semen analyzed using a CASA system to eliminate any operator bias. The patients are then divided into four groups: 1) those with normal semen parameters (WHO 2010); 2) those which are asthenozoospermic/asthenospermic (reduced/<40% sperm motility); 3) those which are oligozoospermic/oligospermic (reduced/<15Million/mL sperm concentration); and 4) those which have <4% morphologically normal sperm. After their initial assessment, each sample is processed using a discontinuous Density Gradient technique (45% and 80% gradients). The prepared sample is again assessed using the CASA system and images and data stored for further evaluation. Any difference observed in the prepared sample is noted. A 1-2 μL aliquot of each prepared sample is then added to 5 μL of polyvinylpyrrolidone (PVP) and the curvilinear velocity and lateral head displacement, are analyzed on the CASA system and images recorded. These provide three individual sets of data for the same sample. All these data are correlated with each other and with each group data.

Percentage of DNA fragmentation in each of the four groups, neat and post washed is measured using a single cell gel electrophoresis assay (COMET assay) and is correlated with DNA fragmentation using a terminal deoxynucleotidyl transferase dUTP nick end labeling assay (TUNEL assay). Other DNA fragmentation assays, the HALO sperm test and the Sperm Chromatin Structure Assay (SCSA), can also be assessed thus giving four independent values for each test.

All the images stored and the values of DNA fragmentation obtained are correlated to the morphology of the sperm, curvilinear velocity and lateral head displacement. A visualized algorithm incorporated within the software manipulation allows the establishment of a database, which can identify a single sperm with <5% DNA fragmentation.

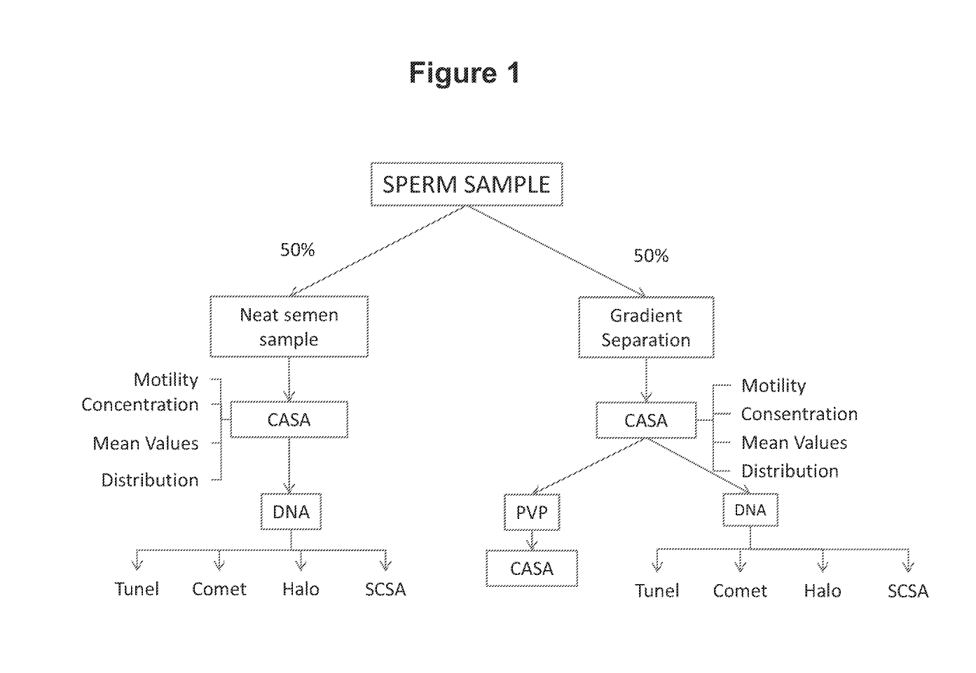

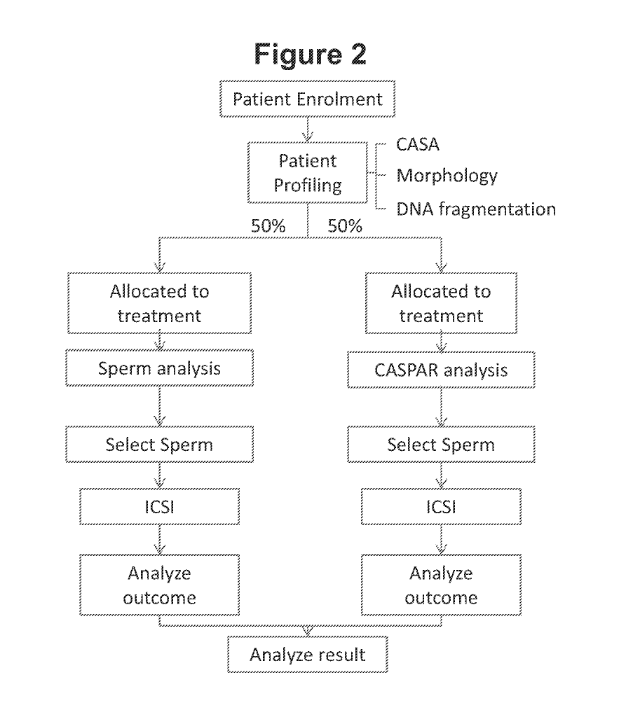

In phase two, a multicenter, prospective randomized study is carried out to show that clinical pregnancy rates with spermatozoa selected using the methods of the invention (e.g., CASPAR) are significantly better than spermatozoa selected using traditional CASA. The study can include approximately 200 patients. During patient enrollment, the patient inclusion criteria are: (1) patient couples undergoing ICSI (male factor indication); (2) first-time treatment; and (3) patients who have undergone no more than 2 previous failures are accepted. The study can be designed as shown in FIG. 1 or FIG. 2. The data is collected and analyzed against number and embryo quality at transfer, clinical pregnancy, and fetal heartbeat by ultrasound, first trimester ultrasound and second trimester ultrasound that picks up unexplained abortions characteristic of DNA fragmentation. This study compares its findings to the current industry diagnostics for semen samples. The purpose is to identify and discriminate between low and high fragmented sperm, and there is no risk associated with the study.

The next challenge is to design the algorithm and device that enables the embryologists to analyze any human semen sample and perform a real-time comparison between the analyzed sample and the reference material in the database to reveal data on DNA fragmentation as well as standard CASA results to minimize the existing risk factors, which can include, but is not limited to, uncertain outcomes as external factors can influence clinical pregnancy rates, development of algorithms for real time comparison of fresh sperm samples with reference database, and reliability and reproducibility of results. The algorithm is shown in FIG. 3.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A method for identifying and selecting high motility, normal morphology and high DNA integrity spermatozoa comprising: (a) receiving a sperm sample; (b) determining one or more characteristics of each spermatozoon in the sperm sample; and (c) determining a sperm kinetic profile of the spermatozoon in the sperm sample; (d) and associating the sperm kinetic profile with an index set, wherein the index includes information indicative of an identity of the sperm kinetic profile.

2. The method of claim 1, wherein determining the sperm kinetic profile comprises determining a fingerprint value at one or more landmarks of the spermatozoon, wherein each landmark represents a distinctive and reproducible component of the spermatozoon, and wherein the fingerprint value represents a number of features of the spermatozoon at the distinctive and reproducible components of a given spermatozoon.

3. The system of claim 1, wherein the spermatozoon has 5% or less DNA fragmentation.

4. The system of claim 1, wherein the spermatozoon is from Homo sapiens.

5. The system of claim 1, wherein the spermatozoon identified to have a preferred kinetic sperm profile is selected for use in a fertilization process.

6. A hardware platform device with an integrated tracking and machine vision system and a pattern matching algorithm that matches the input from the machine vision system with the pattern matching algorithm based on a set of pre-defined patterns in a reference database.

7. The hardware platform device of claim 6, wherein the set of pre-defined patterns recognize sperm with motility and morphology patterns that match profiles with high DNA integrity for use in the fertilization process, and thus significantly increasing the probability of a viable clinical pregnancy.

8. The hardware platform device of claim 6 comprising: a buffer configured to receive a sperm sample; a processor; and instructions stored in memory and executable by the processor to cause the processor to perform functions comprising: receiving the sperm sample into the buffer; storing the received sperm sample; and performing a content identification of each spermatozoon in the buffer to determine a kinetic sperm profile of the received sperm sample.

9. A hardware platform device comprising a handheld system, a microfluidic chip and software and pattern matching algorithms.

10. The hardware platform device of claim 9, wherein the microfluidic chip and software can be used for semen analysis.

11. The hardware platform device of claim 9, wherein the microfluidic chip and software can be used for oocyte analysis.

12. The hardware platform device of claim 9, wherein the microfluidic chip and software can be used for embryo scoring.

13. The hardware platform device of claim 9 comprising a communication device for transfer of data to cloud applications.
